(19) United States
(12) Patent Application Publication
(10) Pub. No.: US 2004/0092786 A1
(43) Pub. Date: May 13, 2004

(54) Intraoperative dynamic dosimetry for prostate implants

(75) Inventors: Marco Zaider, Bronx, NY (US); Dorin Todor, Richmond, VA (US); Michael Zelefsky, New York, NY (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Memorial Sloan-Kettering Cancer Center, New York, NY (US)

(21) Appl. No.: 10/292,383

(22) Filed: Nov. 8, 2002

(51) Int. Cl. A61N 5/00

(52) U.S. Cl. 600/1

(57) ABSTRACT

A method for treating a prostate gland comprises the steps of implanting therapeutic seeds in the prostate gland of a patient; imaging the implanted seeds with an imaging probe; calculating the three-dimensional locations of the implanted seeds and measuring the dosimetry of the implanted seeds; calculating optimal locations for additional seeds to provide an optimal post-implant dosimetric measurement; and implanting additional seeds in the optimal locations. The method may implement computer hardware and software.

### BACKGROUND OF THE INVENTION

The present invention relates generally to the field of brachytherapy and more specifically to an improved method for intraoperatively evaluating the dosimetry for therapeutic implants into a prostate gland.

Depending on the location of the cancer tissue, different types of radiation therapy (“radiotherapy”) may be employed. An externally applied radiation beam (such as through a Cobolt-60 teletherapy machine) is acceptable for treating cancerous tumors in say, for example, the brain. Implanted radioactive bead-like elements, commonly referred to as ‘seeds,’ provide an internally applied (delivered) radiation therapy. Implanted radiation seeds are typically used to treat prostate cancer and in that case the radiotherapy is termed prostate brachytherapy.

The general process for prostate brachytherapy includes anesthetizing the patient such that the patient will have no feeling from the waist down, locating the prostate gland with transrectal ultrasound through a probe device inserted into the rectum, and placing the seeds within the prostate gland in a sequential manner by inserting needles carrying the seeds just behind the scrotum.

Currently, the quality of the implant is very much dependent on the physician's skill and experience. The implant can be evaluated only after the procedure is completed with no possibility for remedial action. The method of the present invention provides the physician with a tool for real-time (i.e. during the actual implant) feedback on the dosimetry of the seeds implanted. This allows the physician to re-optimize the plan (to the extent that seeds were not implanted at the prescribed positions), and at the end verify—with the patient still in the operating room—that the prescribed prostate coverage was indeed obtained. When this is not the case, additional radioactive sources may be implanted.

Over the last 5 to 10 years, there has been a resurgence of interest in the use of permanent prostate implantation for patients with clinically localized prostate cancer. With the development of improved techniques using CT-based (CT=Computerized Tomography) or ultrasound-based transperineal (TRUS) techniques, the published results of PSA (PSA=prostate specific antigen) control for patients with early stage prostate cancer treated with this modality have shown significant improvement compared to the open retropubic technique (Zelefsky, M. J., Y. Yamada, G. Cohen, E. S. Venkatraman, A. Y. C. Fung, E. Furhang, D. Silvern, and M. Zaider (2000) Postimplantation dosimetric analysis of permanent transperineal prostate implantation: improved dose distribution with an intraoperative computer-optimized conformal planning technique. Int. J. Radiat. Onc. Biol. Phys. 48: 601-608) and are in the range of 80-90% at 5 years or longer (Beyer, D. C., and J. B. Priestlry (1997) Biochemical disease free survival following 125-I prostate implantation. Int. J. Radiat. Oncol. Biol. Phys. 37: 559-563; Blasko, J. C., H. Ragde, R. W. Luse, J. E. Sylvester, W. Cavanagh Frimm, P D (1996) Should brachytherapy be considered a therapeutic option in localized prostate cancer? Urol. Clin. North. Am. 23: 633-650; Zelefsky et al, 2000).

Despite the improvement in implant techniques, the approaches that have been used for planning and for assessing the quality of implantation vary from one center to another. Furthermore, current implantation techniques have not incorporated sophisticated computational tools that would allow real-time re-adjustment of overall dosimetry as a result of seed movement, needle distortion and edema during implantation process. There are possible deviations from the ultrasound plan to what is actually observed intra-operatively (Narayana, V., P. L. Robertson, R. J. Winfield, and P. W. McLaughlin (1997) Impact of ultrasound and computed tomography prostate volume registration on evaluation of permanent prostate implant. Int. J. Radiat. Oncol. Biol. Phys. 39: 341-346). In addition, inaccuracies in seed placement can lead to “cold” spots in parts of the target volume and/or “hot” spots in the urethra (urethra complication rates are higher for brachytherapy than for external beam radiotherapy).

With the advent of intra-operative optimized planning, the treatment of prostate cancer with permanent implants has reached an unprecedented level of dose conformity. At Memorial Sloan-Kettering Cancer Center (MSKCC) an intraoperative conformal optimization and planning for ultrasound-based prostate implants, which obviates the need for pre-planning, has been developed and successfully implemented (Lee, E. K., R. Gallagher, and M. Zaider (1999) Planning implants of radionuclides for the treatment of prostate cancer: an application of mixed integer programming. Mathematical Programming Society Newsletter 61: 1-10; Lee, E. K., R. J. Gallagher, D. Silvern, C. S. Wuu, and M. Zaider (1999) Treatment planning for brachytherapy: an integer programming model, two computational approaches and experiments with permanent prostate implant planning. Phys. Med. Biol. 44: 145-165; Lee, E. K., and M. Zaider (2001) Intraoperative iterative treatment-plan optimization for prostate implants. 2nd International Innovative Solutions for Prostate Cancer Care Meeting. 32-33; Silvern, D., E. K. Lee, R. J. Gallagher, L. G. Stabile, R. D. Ennis, C. R. Moorthy, and M. Zaider (1997) Treatment planning for permanent prostate implants: genetic algorithm versus integer programming. Med. Biol. Eng. Comput. 35: 850; Zaider, M., M. J. Zelefsky, E. K. Lee, K. L. Zakian, H. I. Amols, J. Dyke, G. Cohen, Y. C. Hu, A. K. Endi, C. S. Chui and J. A. Koutcher, Treatment planning for prostate implants using magnetic resonance spectroscopy imaging. Int. J. Radiat. Onc. Biol. Phys. 47:1085-1096 (2000). At the time of implant, patients are placed in the extended lithotomy position and a urinary catheter is inserted. An ultrasound probe is positioned in the rectum, and the prostate and normal anatomy are identified. Needles are inserted through the perineal template at the periphery of the prostate. The prostate is subsequently scanned from apex to base, and these 0.5 cm images are transferred to the treatment planning system using a PC-based video capture system. On the computer monitor, the prostate contours as well as the urethra are digitized on each axial image. Needle positions are identified on each image, and their coordinates are incorporated into a genetic algorithm optimization program (Silvern et al, 1997; Zaider et al, 2000; Zelefsky et al, 2000). This sophisticated optimization system incorporates acceptable dose ranges allowed within the target as well as dose constraints for the rectal wall and urethra.

Nevertheless, because of well-documented (and unavoidable) inaccuracies in seed placement into the gland, carrying out a plan results in a large degree of variability relative to the intended dose distribution. FIG. 1 shows a chart 100 which illustrates the magnitude of the problem by comparing, for a random group of 17 patients treated at MSKCC, the percentage urethral volume covered by 150% of the prescription dose (planned 102 (medium gray); assessed at post-implant evaluation 104 (dark)) and also 200% of the prescribed dose (post implant evaluation 106 (white); the planned percentage volume was always zero).

There are significant limitations to conventional methods of prostate brachytherapy treatment planning. Recognized limitations of the pre-planning method (planning performed several days to weeks prior to the procedure) include the fact that often such approaches will not simulate the exact conditions and geometry of the gland as seen in the intraoperative setting. Post-implant evaluations of permanent prostate implants often indicate significant differences between the intended plan and its actual implementation. Several reports in the literature (Dawson, J. E., T. Wu, T. Roy, J. Y. Gu, and H. Kim (1994) Dose effect of seeds displacement deviations from pre-planned positions in ultrasound guided prostate implants. Radiother. Oncol. 32: 268-270; Yu, Y., F. M. Waterman, N. Suntharalingam, and A. Schulsinger (1996) Limitations of the minimum peripheral dose as a parameter for dose specification in permanent I-125 prostate implants. Int. J. Radiat. Biol. Phys. 34: 717-725; Willins, J., and K. Wallner (1997) CT-based dosimetry for transperineal I-125 prostate brachytherapy. Int. J. Radiat. Onc. Biol. Phys. 39: 347-353; Zelefsky et al, 2000) indicate the target coverage with 100% of the prescription doses is often less than 90% and the dose to normal tissues such as the urethra and rectum are highly variable. While an experienced physician can lessen the magnitude of these differences, many factors controlling execution of the plan are subject to random fluctuations. It has recently been reported that an intraoperative 3D conformal (I-3D) planning system, currently in use at MSKCC, has addressed some of these important concerns (Zelefsky et al, 2000).

A case in point are urethra-related complication rates which are considerably higher for brachytherapy than for teletherapy. Larger than planned doses to the urethra can have significant consequences in terms of late urinary toxicity. The dosimetric parameters and acute urinary toxicity of intraoperative (I-3D) conformal treatments have been analyzed in 182 patients with localized prostate cancer and compared their outcome to 247 patients previously treated at MSKCC with a pre-planned CT-based implantation approach. Table 1 below shows the incidence of acute grade-2 urethral toxicity during the first 6 months and from 6 to 12 months after the procedure, as well as the average urethral dose (expressed as percentage of the prescription dose) for the two types of treatments.

TABLE 1
The incidence of acute grade-2 urinary toxicity

| Technique | \overline{D}ᵤᵣₑₜₕᵣₐ/D_{prescription} | <6 months | 6-12 months |
|---|---|---|---|
| Pre-planning | 182% | 85% | 58% |
| I-3D | 143% | 46% | 23% |
| p-value | | 0.01 | 0.01 |

It was also noted that lower urethral dose had an impact on the time of resolution of acute grade 2 urinary symptoms at 6 months after the procedure. Among those patients with maximal urethral <200%, the likelihood of symptom resolution at 6 months was 42% compared to 21% among those patients with maximal urethral doses >200% (p=0.05).

Nevertheless, there remains continued room for improvement to enhance accuracy of the brachytherapy procedure and further reduce dose to normal tissue structures.

### SUMMARY OF THE INVENTION

In order to best address the problems outlined above, it would be important to obtain real-time dosimetric information during the seed implantation procedure so that—based on the actual positions of the seeds already implanted—the treatment can be modified by re-optimizing the plan in mid-procedure. With the same procedure the quality of the implant at the end of the procedure can be assessed. If changes are needed, they can be performed immediately. Applicants also suggest replacing the so-called “post-implant evaluation” (usually CT) by the “operating room evaluation” performed with the present application. This brings important benefits to the patient and reduces costs.

Periodic real-time readjustment of the intraoperative plan—a procedure hereinafter referred to as intraoperative dynamic dosimetric optimization (IDDO)—provides a significant improvement in implant dosimetry and accordingly in clinical outcome. A phase-II clinical trial is designed to assess whether implantation with IDDO leads to sufficient decrease in urethral toxicity to warrant further (randomized) studies.

According to the present invention, IDDO comprises:

1. Implanting seeds into a patient.

2. Software (or a computer-implemented process/methods) that automatically reconstructs seed coordinates from images (for example, fluoroscopic images) taken at three different angles. The computer process identifies seeds intraoperatively (i.e. in real time during the medical procedure) in each image, matches them and calculates their coordinates in a patient coordinate system (for example, the fluoroscopy imaging coordinate system (FCS)).

3. An algorithm for registering information from the FCS into a planning coordinate system (for example, the ultrasound imaging coordinate system (UCS)). Contours of the prostate and other structures are outlined by the physician in the UCS, which is referenced with respect to the ultrasound (US) probe. To fuse the two spaces, a set of non-coplanar reference points (lead markers) that can be identified both in the FCS and in the UCS are attached to the US probe. A minimum of four such points is needed. This uniquely determines the transformation between the two coordinate systems, allowing one to register anatomical structures (prostate, urethra, etc.) on seed locations.

4. Automated planning software that, with current partial dose information based on seeds actually implanted, re-optimizes the plan to account for inaccuracies.

In the preferred embodiment, the entire procedure (acquisition of fluoroscopic images, seed localization, image registration and re-planning) is expected to take less than 10 minutes.

Because of the increasing number of centers performing prostate implants in the United States, the potential significance of IDDO may be far-reaching. The resultant outcome of the seed placement is often suboptimal in terms of areas of tumor underdosage and/or unnecessarily high doses delivered to normal tissues. These concerns will result in higher likelihood of tumor recurrence and/or increased treatment-related toxicity. The viability of being able to re-optimize in real time also allows modification of plans due to unforeseen difficulties during an operation. These modifications can potentially correct any areas of tumor underdosage prior to completion of the procedure. In addition, the operator will become more cognizant what the real-time dose to the urethra and rectum is, and dynamic adjustment of the intraoperative plan can be made to insure that the final dose delivered to these structures remains as low as possible.

The implementation of IDDO makes use of standard equipment that is found in any operating room and thus there is virtually no additional cost associated with this procedure. Finally, IDDO may obviate the need for post-implant CT-based evaluation, as the entire prostate evaluation can be performed at the end of the medical procedure using ultrasound and fluoroscopic fusion/registration.

A method of the present invention for implanting therapeutic seeds in a target tissue comprises the steps of (a) placing (implanting) a first set of therapeutic seeds in the target tissue, (b) imaging the implanted seeds in the target tissue to produce two-dimensional images of the implanted seeds, (c) determining the three-dimensional coordinates of the seeds using a computer-implemented method, (d) calculating the dosimetry of the implanted seeds based on their determined locations, (e) determining optimal locations in the target tissue for additional seeds to be implanted such that the total dosimetry of the implanted seeds and the additional seeds optimizes the therapeutic effect on the target tissue, and (f) implanting the additional seeds in those determined optical locations. These steps may all occur within a single operative session.

The step of imaging may include ultrasound imaging, fluoroscopic imaging or magnetic resonance imaging. Further, the step of imaging the implanted seeds may include obtaining three or more two-dimensional images, each from a different angle. Likewise, the step of determining the three-dimensional locations of the implanted seeds may include evaluating seed projections on three or more two-dimensional images.

The method may also include the step of translating seed locations from a patient coordinate system to a planning coordinate system and this step may include the use of lead markers located on an ultrasound probe. The patient coordinate system may be a fluoroscopic imaging coordinate system and the planning coordinate system may be an ultrasound imaging coordinate system. The translating step may include the steps of identifying at least two common markers in each of the two coordinate systems, measuring the coordinates in the planning coordinate system of the markers with respect to a template, reconstructing those measurements from their two-dimensional projections in the patient coordinate system, and calculating a general transformation which when applied to the markers will result in the superposition of the two sets of markers, i.e. the transformation will allow the coordinates of the markers in one of the coordinate systems to coincide with the coordinates of the markers in the other coordinate system.

The step of determining three-dimensional locations of implanted seeds may include, (i) for a given seed in a first image, identifying seeds in the second and third images that are within an interval Dy from the given seed (i.e. identifying candidates which may be images of the same seed as the given seed), (ii) generating seed triplets comprised of the given seed and the identified seeds, (iii) calculating, for each seed triplet, positions of virtual seeds for generating the first, second and third images of the seeds in the triplet (i.e. where the seeds would have to be in the first image to generate the projection of those seeds in the second and third images), (iv) determining distances between the calculated positions of the virtual seeds, said determination including projecting positions of virtual seeds into an x-z plane (y=0), (v) ranking the seed triplets relative to each other based on the calculated positions of the virtual seeds, (vi) selecting, based on the ranking, a seed triplet to represent the given seed, and (vii) repeating these steps for each seed in the first image. The first step may also begin with choosing a given seed from the second or third images and identifying seeds in the other two images to generate seed triplets.

These determinations, calculations, ranking and selection may be made by computer implementation.

The method may also include calculating a y-coordinate for each virtual seed in the triplet and ranking the seed triplets based on calculated y-coordinates.

The step of determining the calculated positions of the virtual seeds may further include the steps of assigning to certain seed triplets a respective average virtual seed defined by an average of x, y, z coordinates of the virtual seeds of the triplet, projecting the respective average virtual seeds back on the first, second and third images, measuring the distances between the projected seeds and the respective given and identified seeds, and ranking the seed triplets based on smallest measured distance.

The method may further comprise the selection of a “true” seed to represent the given seed based on the multiple aforementioned rankings.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a chart showing the percentage urethral volume typically covered by the stated percentage of the prescription dose.

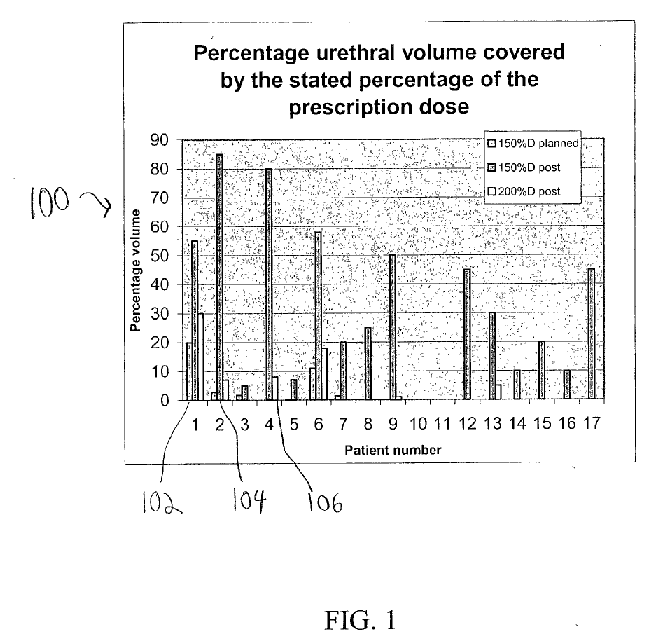

FIGS. 2A-2C are fluoroscopic images of a seed implant with lead markers, where lead markers alone are shown in FIG. 2B, implanted seeds alone are shown in FIG. 2C, each of which was automatically segmented from the image in FIG. 2A.

FIG. 3 shows a histogram of seed clusters areas, one of the tools, which allows the separation of single seeds from clusters of multiple seeds.

FIG. 4 is an example of clusters (labeled by numbers) automatically detected on the fluoroscopic image in the preferred embodiment.

FIG. 5 illustrates automatic detection and 3D reconstruction of 75 seeds imbedded in a prostate phantom.

FIGS. 6A-6C are post-implant isodose curves for a pre-implant plan (FIG. 6A), a real-time plan (FIG. 6B), and a real-time IDDO plan (FIG. 6C) of the present invention.

FIG. 7 is a schematic illustration of a two-film seed reconstruction.

FIGS. 8A-8C show typical seed projections for a ¹⁰³Pd implant for which the present invention is able to reconstruct seed coordinates.

FIG. 9 illustrates a three-film seed reconstruction as employed by the preferred embodiment.

FIGS. 10A-10B are schematic diagrams of a three-film seed reconstruction, with an enlarged view of one portion shown in FIG. 10B.

FIGS. 11A-11C show films used in the assignment of seed triplets in the present invention.

FIG. 11D is a flowchart showing the steps for identifying seeds in the images.

FIGS. 12A-12C illustrate reconstructed and digitized seeds according to the present invention.

FIG. 13 is a perspective view of a typical pelvic phantom.

FIGS. 14A and 14B are side views of a prototype ultrasound probe and an enlargement of the tip of the probe, respectively.

FIG. 15 is a schematic view of an operating room in which the methods of the preferred embodiment are implemented.

FIG. 16 is a flowchart describing the overall operative procedure of the preferred embodiment.

### DETAILED DESCRIPTION OF THE INVENTION

A description of preferred embodiments of the invention follows.

The invention IDDO process begins in the middle of the operation, i.e. after some therapeutic (radioactive) seeds have already been implanted into the target tissue, i.e. prostate gland. IDDO comprises the steps of detecting the implanted seeds, reconstructing or mapping the implanted seeds into a three-dimensional coordinate system, transformation of the seed locations from a patient coordinate system to a planning coordinate system, and reoptimizing the implant locations for remaining seeds to be implanted. The planning coordinate system can be an ultrasound image coordinate system (UCS) and the patient coordinate system can be a fluoroscopic image coordinate system (FCS). The methods and examples discussed herein will refer to UCS and FCS, but it is contemplated that other imaging methods and coordinate systems can also be employed.

The IDDO process will first be described in the context of preliminary experimentation and then a more detailed description of the individual steps of the process will be presented.

Automatic Seed Detection:

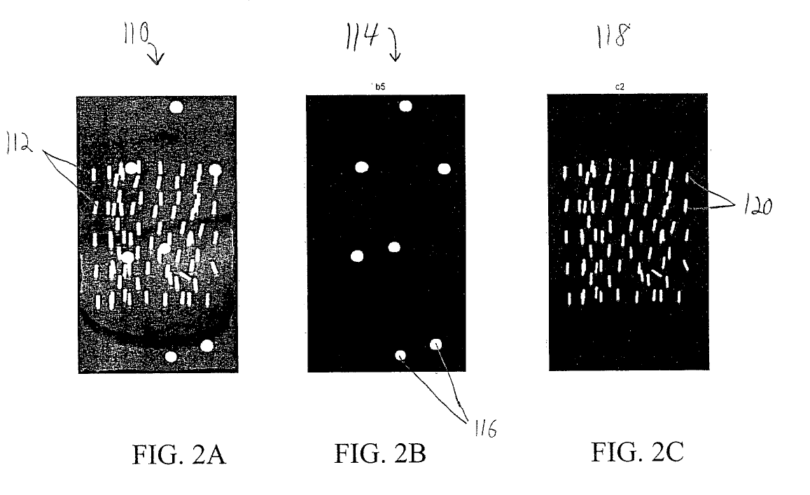

A preliminary algorithm for seed segmentation (identification) was developed using MATLAB, the Image processing Toolbox (Mathworks, Inc.), and the SDC Morphology Toolbox (SDC Information Systems). The main steps are illustrated in FIGS. 2A, 2B and 2C. First, a gray-level image 110 (FIG. 2A) of the implanted seeds 112 is acquired, either by scanning the radiologic film or by acquisition of a fluoroscopic image. Next, from the gray-level image 110, a binary image 114 (FIG. 2B) of the lead markers 116 is extracted. The coordinates of the lead markers 116 are computed using known techniques (the lead markers 116 are attached to the ultrasound probe (not shown) and are used for registering information from the fluoroscopic imaging coordinate system to the ultrasound imaging coordinate system—discussed further below). Third, the seeds 120 in the gray-level image 110 are segmented (i.e. identified) using shape recognition algorithms, separating single seeds from clusters of seeds, and resolving clusters into individual seeds. The segmented seeds 120 form working binary image 118 (FIG. 2C) and the corresponding seeds 120 location and orientation information is subsequently used in the 3D reconstruction.

The process of segmentation is commonly ill posed because partitioning an image may result in regions that do not necessarily correspond to complete objects. This is further complicated by the fact that one deals with the 2D projection of a 3D object and this makes shape recognition difficult to implement in a user-independent computer-automated mode.

A pelvic phantom (described below), with 75 dummy seeds implanted in the prostate and an image probe placed in its rectal space, was imaged with a C-arm fluoroscopic unit (gantry at 0⁰). The original gray-level image 110 and the resulting (automated) lead marker binary image 114 and seed segmentation binary image 118 are shown in FIGS. 2A-2C.

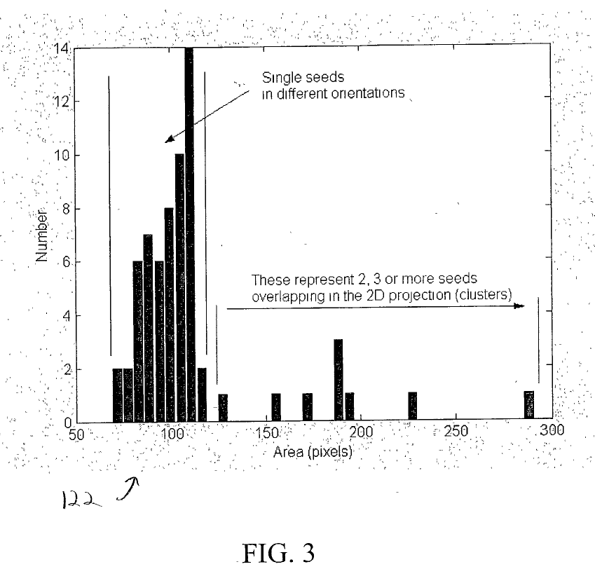

In the binary image 118 that contains only seeds 120 (FIG. 2C), clusters of seeds are separated from single-seed images by measuring for each blob (cluster) three parameters: the area, its perimeter and its eccentricity. Together, deviations from the average value of the three parameters are used to identify “uncertain regions”. FIG. 3 shows a histogram 122 with respect to area of blobs in a working seed image 118. In the histogram 122, single seeds that cover up to just over 100 pixels in area define the “normal” seeds. Clusters of 2 or more overlapping seeds that cover areas of about 125 pixels to just under 300 pixels are shown as abnormal seeds (or clusters).

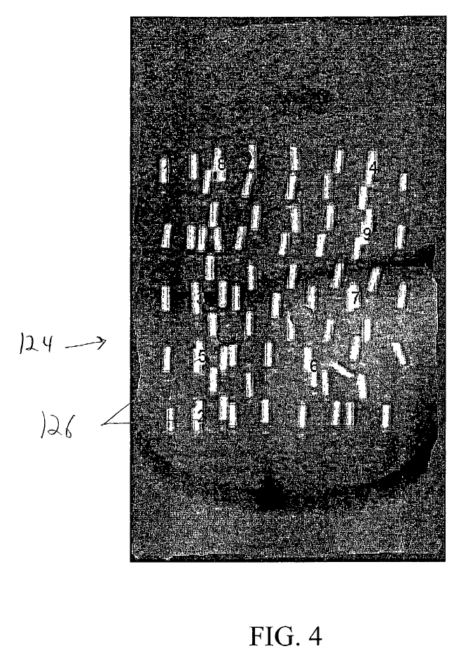

FIG. 4 shows a fluoroscopic image in which detected seed clusters 126 have been identified with numbers 1-9. These regions are next subjected to a more elaborate analysis in which shape is discriminated. In the preferred embodiment, this is accomplished by calculating a feature vector of each blob (cluster).

A variable (also known as gestalt variable) (π)₂—useful in describing and ordering different shapes is defined in terms of the perimeter P and area A of a subject blob (detected seed cluster): (π)₂=P²/4A [for a circle (π)₂=π.]

To retain invariance to rotations, the sorting algorithm uses the moment invariants as defined by Hu, M. K. (1962) Visual pattern recognition by moment invariants. IRE Tr. on Inf. Theory 8: 179-187:

Φ₁=v₂₀+v₀₂

Φ₂=(v₂₀−v₀₂)²+4v₁₁²

Φ₃=(v₃₀−v₁₂)²+(3v₂₁−v₀₃)²

Φ₄=(v₃₀+v₁₂)²+(v₂₁+v₀₃)²

Here v_{pq} is the generalized central moment of order (p+q)

$${v}_{pq} = \frac{{μ}_{p q}}{{μ}_{00}^{w}} , with w = \frac{p + q}{2}$$

and μ_{pq}=∫∫(x−x_{cg})^{p}(y−y_{cg})^{q}dxdy

where (x_{cg},y_{cg}) are the coordinates of the center of gravity.

In the present invention, for each subject blob (seed cluster), the area, perimeter, number of end points, eccentricity, gestalt variable (π)₂ and the four moment invariants Φ₁₋₄ are calculated. These calculated features are placed into a feature vector, which is used for cluster sorting. By analyzing a large number of real cases, heuristic rules have been identified for recognizing each type of cluster (e.g. two seeds in parallel, two seeds overlapping along the major axis). Using the end points of the cluster skeleton, respective centers of the seeds that make up the cluster can now be calculated.

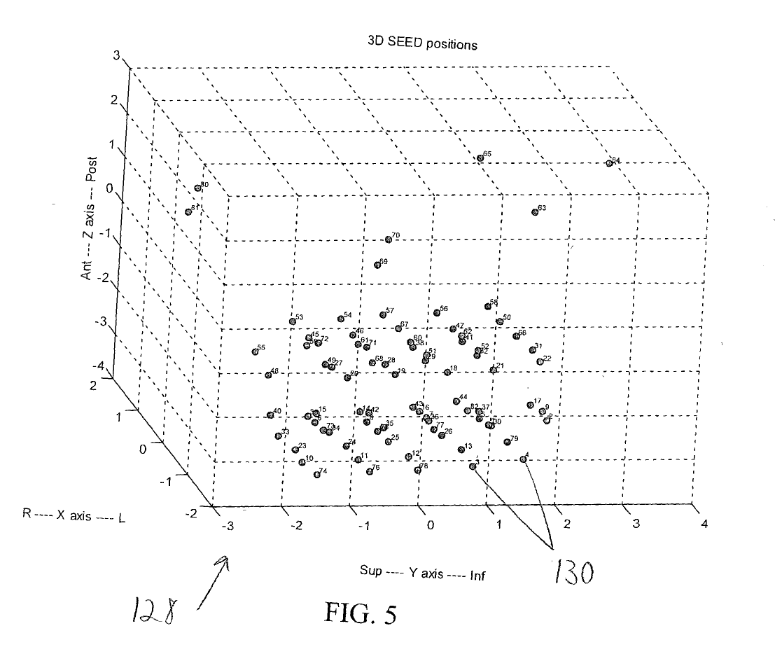

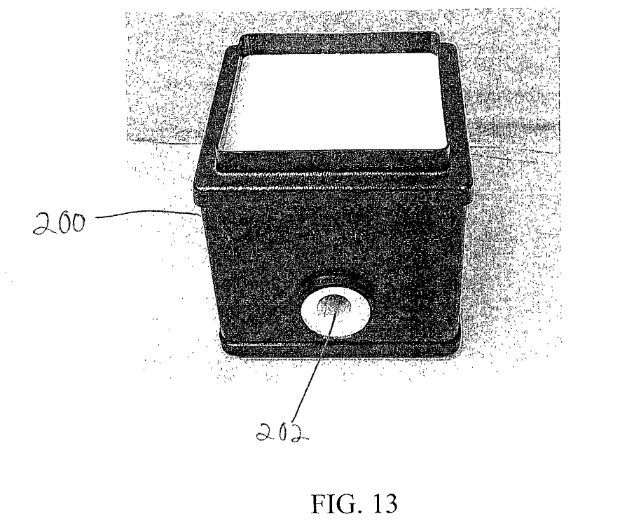

FIG. 5 illustrates the three-dimensional plot 128 resulting from automatic detection and 3D reconstruction of seventy-five seeds 130 imbedded in a prostate phantom 200 (FIG. 13). Three fluoroscopic images (at 0⁰,±15⁰), were used in this analysis.

Validation of Seed Coordinate Reconstruction and the Coordinate Transformation Tools from FCS and UCS:

A CT scan of the prostate phantom 200 (FIG. 13) with prototype ultrasound probe 204 (discussed below) was performed and the positions of the seventy-five dummy seeds 130 imbedded in phantom 200 plus the x-ray lead markers 208 attached to the ultrasound probe 204 were determined using a commercial post-implant analysis software package (INTERPLANT V.1.0, Burdette Medical Systems, Champaign Ill.). Three fluoroscopic images of the same system were also taken. The x-ray markers 208 and dummy seeds 130 were identified and their positions reconstructed. Using the x-ray markers 208 as a guide, the seed 130 coordinates were transformed to the CT space taken here to simulate the UCS. Seed coordinates (in plot 128 of FIG. 5) thus obtained were found to be in excellent agreement with coordinates obtained in the CT study.

Re-Optimization in IDDO Procedure:

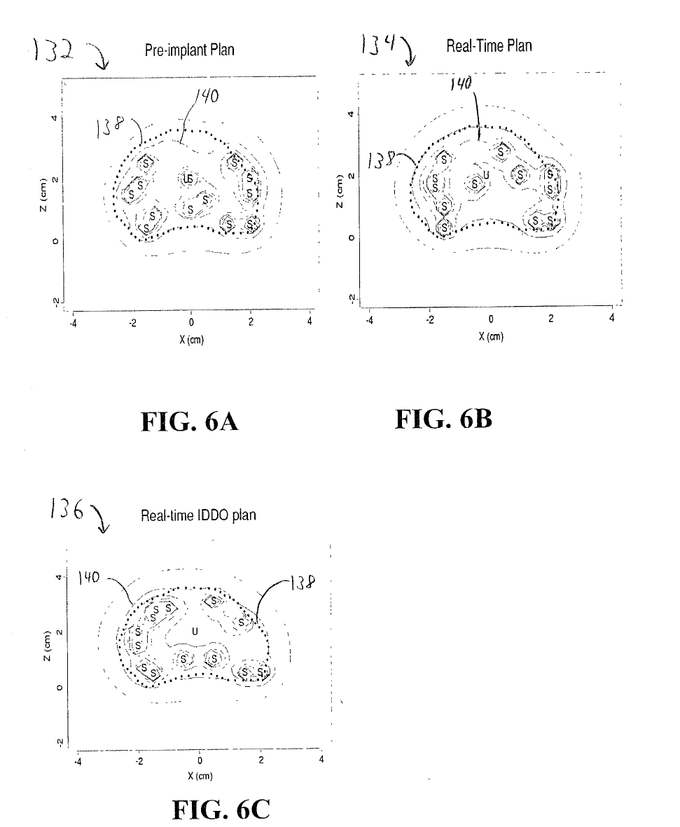

Preliminary findings are illustrated using the re-optimization step in the invention IDDO procedure for a patient case. FIGS. 6A-6C show post-implant isodose curves 132, 134, and 136, respectively, for each of the three plans (pre-implant plan (FIG. 6A), at the time of implantation (FIG. 6B), and with IDDO re-planning (FIG. 6C)) for the same slice of the subject gland. For this patient, radioactive beads formed of ¹²⁵I and a target dose of 144 Gy (TG43) is prescribed. In each of FIGS. 6A-6C, the bold black dotted line 138 outline the contour of the prostate, and the solid curve 140 closer to the prostate contour represents the 100% prescription isodose. Implanted seeds are marked ‘S’ and the urethral region is labeled ‘U’. The ideal situation is for the prescription isodose curve 140 to wrap perfectly around the prostate contour 138, which would indicate both perfect coverage and perfect conformity. This is seldom achievable in practice. The optimized plans of the present invention strive to cover the entire prostate boundary 138 while avoiding excess irradiation to external normal tissue.

Turning now to FIG. 6A, for the plan designed prior to implantation, the post-implant coverage is only 75% with a hot spot in the urethral region where seeds ‘S’ are shown close to or overlapping urethral region ‘U’. This can be explained by the fact that there is a discrepancy in the prostate volume and organ position during simulation and during implantation. A pre-implant plan can only provide a crude estimate of the final seed configuration used for the actual treatment.

The real-time plan (FIG. 6B), which is designed based on the ultrasound images taken during the operation, results in a dose coverage of 94%, while offering a low conformity value (1.24). The urethral dose while better than in the pre-implant plan of FIG. 6A, is between 100% and 160% of the prescribed dose. In contrast, with the IDDO plan (FIG. 6C), 100% post-implant coverage is achieved while urethra dose is maintained within the desired 100% to 120% prescription dose range, and rectum dose is kept below 78% of prescription dose.

This preliminary result suggests that IDDO treatment planning offers a practical way of achieving post-implant dosimetry goals, including improved post-implant dose coverage and low irradiation to urethra. With real-time treatment planning, it is possible to dynamically re-optimize treatment plans to account for actual seed positions (as opposed to planned positions) and needle induced swelling to the gland during implantation.

Preliminary Clinical Data:

Preliminary studies have demonstrated that intraoperative planning techniques for prostate brachytherapy using inverse planning and a genetic algorithm for optimization were associated with improved target coverage with the prescription dose and decreased urethral and rectal doses. In the following comparative analysis, lower urethral doses achieved with these approaches have significantly impacted upon the quality of life of treated patients with lower acute toxicity profiles after treatment.

Between 1998 and year 2000, 253 patients were treated with ultrasound-based I-125 implantation using intraoperative 3-dimensional conformal optimization (I-3D). Serial ultrasound images with needles peripherally placed within the prostate were captured intraoperatively, and prostate and normal tissue structures were subsequently contoured. An inverse-planning optimization program was used to identify the optimal seed-loading pattern taking into account pre-determined dose volume constraints for the target, urethra and rectum. After the optimization and the dose calculations were completed, isodose displays were superimposed on each transverse ultrasound image and carefully evaluated. The implant dose was 144 Gy (TG-43) which was prescribed to the isodose line which encompassed the prostate. Clinical stage was as follows: T1C-185 (78%), T2a-48 (20%), T2b-5 (2%). Androgen ablation 3 months prior to implantation for the purpose of volume reduction was used in 92 (39%) patients. The median PSA was 6 ng/ml. The median follow-up was 24 months and the minimum follow up was 6 months. Dosimetric parameters (based on the post-implantation CT evaluations) as well as acute toxicity in these patients were compared to 247 who were previously treated at MSKCC with pre-planned CT based transperineal I-125 implantation.

For the I-3D group, the V100 (percent prostate volume receiving 100% of the prescription dose) and D90 (percent dose delivered to 90% of the prostate) were 96% and 120%, respectively. The average urethral dose was 143% of the prescription dose. As already mentioned, reduced urethral doses associated with this intraoperative 3D approach resulted in a significant reduction in acute toxicity in these patients observed during the first year after implantation. The need for alpha-blocker medications to control grade 2 urinary symptoms during the first 6 months and from 6 to 12 months after the procedure was 46% and 23% respectively. Only 2% experienced grade-3 urinary toxicity with this approach. These dosimetric parameters and tolerance profile were significantly better than the cohort of patients treated with a pre-planned implant approach. Among patients treated with this latter approach the V100 and D90 were 88% and 94%, respectively. The average urethral dose was 182% of the prescription, and the incidence of grade 2 urinary symptoms during the first 12 months after the procedure was 58% (p<0.01).

These data demonstrate that intraoperative conformal optimization for prostate brachytherapy has consistently achieved excellent target coverage with the prescription dose and decreased urethral dose. This favorable therapeutic ratio has resulted in decreased grade 2 urinary toxicity compared to a cohort of patients previously treated at MSKCC with a conventional pre-planned technique. More importantly, these observations represent proof of principle that more optimal dosimetric implant outcome can have a tangible impact on the symptom profile of treated patients and their quality of life. These intraoperative techniques, however, could not achieve dynamic dosimetric optimization with real-time modifications. The invention procedure described below can achieve these goals.

The general scheme for performing the invention IDDO consists of three steps: (1) at several time intervals during the implant, coordinates of implanted seeds are identified, (2) seed images are projected onto the reference frame of the ultrasound images for planning, and (3) the plan is re-optimized. Discussed next are the methodologies and techniques employed in each of these three steps in the preferred embodiment of the present invention.

Quasi-Automatic Seed-Coordinate Reconstruction:

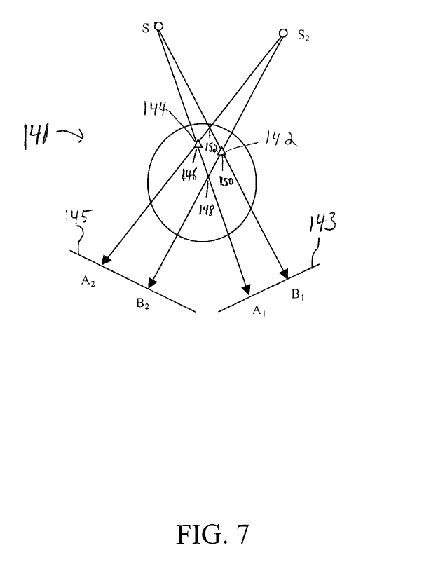

Reconstruction of seed coordinates, for treatment planning or for post-implant evaluation of the dose distribution, is a common and unavoidable task in brachytherapy. It is generally carried out by using planar images taken (isocentrically) at several angles (Amols, H. I., and I. I. Rosen (1981) A three-film technique for reconstruction of radioactive seed implants. Med. Phys. 8: 210-214; Siddon, R. L., and L. M. Chin (1985) Two-film brachytherapy reconstruction algorithm. Med. Phys. 12: 77-83; Biggs, P. J., and D. M. Kelley (1983) Geometric reconstruction of seed implants using a three-film technique. Med. Phys. 10: 701-704). The nature of the problem may be understood from FIG. 7, which shows a two-film 143, 145 seed reconstruction 141. Let A1 and B1 be the projections of two seeds (represented here by the triangles 144 and 142 inside the sphere) when the light source occupies position S1. Similarly, A2 and B2 are projections of the same two seeds when the light source occupies position S2. Given four such projections (hereafter referred to as 2D seed coordinates), but no information as to which projection (A1, B1, A2, B2) belongs to which seed 142, 144, there are four possible reconstructed seed positions corresponding to the four intersections 146, 148, 150 and 152 shown in FIG. 7. To obtain the correct positions from the two images 143, 145 (here a total of four 2D points), one needs to establish a priori that projections (A1,A2) represent a particular seed, and likewise for (B1,B2). As a rule, visual (manual) matching pairs of seed projections that belong to the same seed are identified by a comparable superior-inferior coordinate (along the y-axis) and/or by their relative orientation.

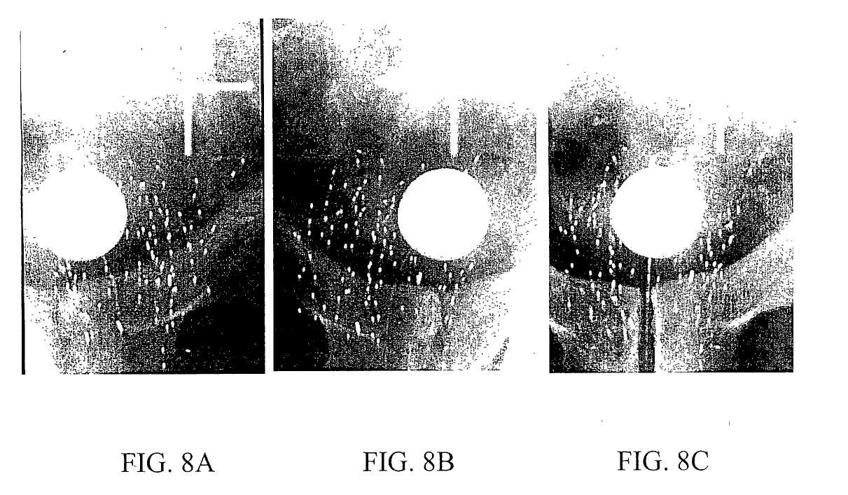

FIGS. 8A-8C show typical seed projections (at 0°, 20° and 340°) for a ¹⁰³Pd implant. For an actual implant (as seen in FIGS. 8A-8C), seed reconstruction is a time-consuming activity and the result is often inaccurate, the latter being a consequence of various objective (the y coordinates of, say, A1 and A2 are not expected to be exactly the same) or subjective factors such as patient motion, seed overlap, poor images, or several seeds with the same y coordinate. The cross is the projection of the rotation's isocenter point and the bright disc is simply the projection of a Foley balloon inserted in the bladder and filled with a contrast substance.

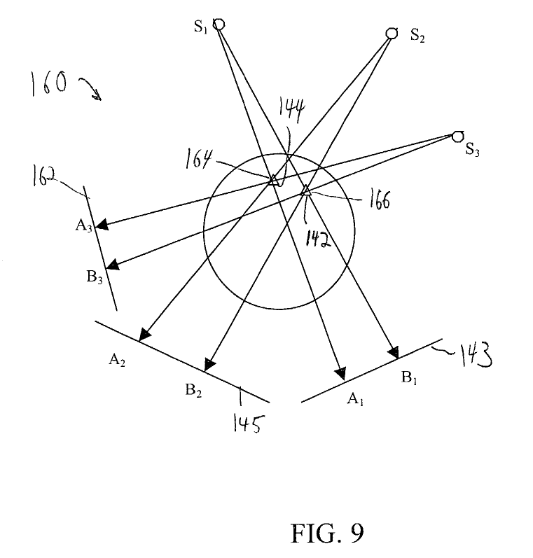

FIG. 9 is a schematic illustration of a three-film seed reconstruction 160 as utilized by the present invention. A third planar image 162 eliminates the ambiguity of the two image 143, 145 approach shown in FIG. 7. In the three film 143, 145, 162 case, the intersection points 164 and 166 of three lines formed by the projections uniquely determines position of each seed 142, 144. In FIG. 9, intersection 164 is formed by and hence is said to correspond to the three lines (A1S1, A2S2, A3S3) for seed 144 in position A. Similarly, intersection 166 corresponds to lines (B1S1, B2S2, B3S3) for seed 142 in position B. However, the identity of the triplet of points (e.g. A1, A2, A3) that represent one seed remains unknown and, in principle, one would have to examine all possible combinations of triplets. Nevertheless, in practice one can take advantage of the fact that projections that belong to the same seed are, along the y-axis, within close proximity and thus investigate only projections that are within an interval Dy of any initial guess.

Algorithm:

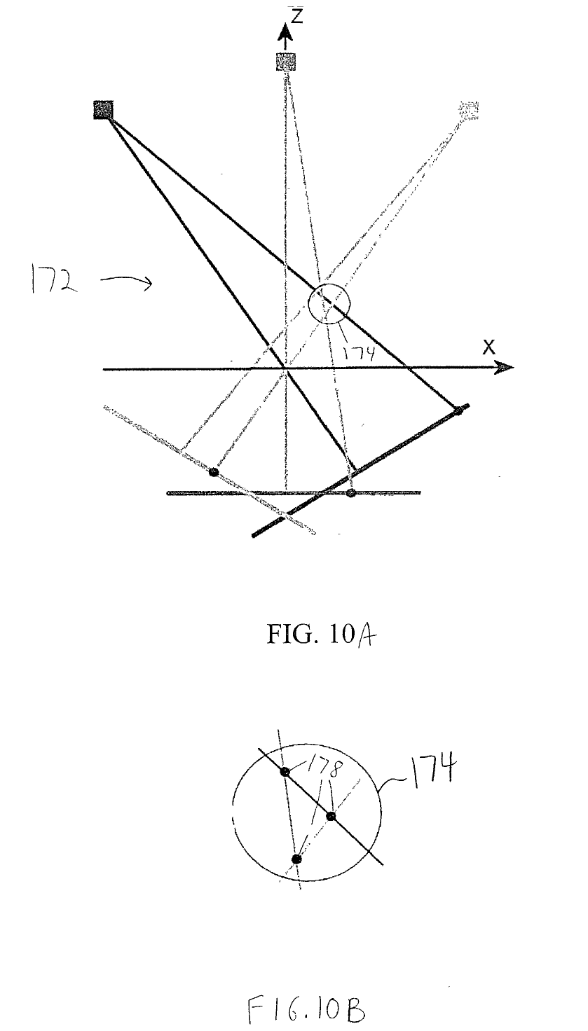

In practical situations relatively small errors in the 2D seed coordinates (i.e. in the planar images) would make the problem ill-conditioned, which means that no three back-projection lines will actually intersect in exactly one point. FIG. 10A shows a diagram 172 of a three-film seed reconstruction with a circle 174 around the area where three such lines are close to intersecting each other but do not. FIG. 10B is an enlarged view of the circled area 174 of FIG. 10A and shows the seeds 178 at respective intersections of two lines each. Applicants employ a set of ad hoc rules (in a sense, a learning algorithm) that attempts to minimize—in the presence of experimental error—seed position misclassifications. This system is fast (less than a minute of PC time) and robust; however, it is not entirely foolproof: occasionally, ambiguities caused by seed overlap must be resolved manually by checking the accuracy of the automatic seed detection.

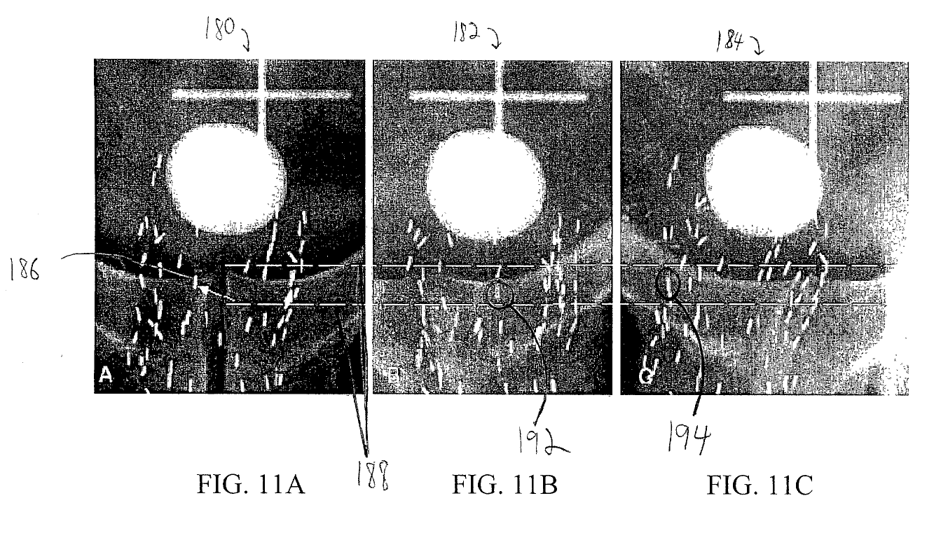

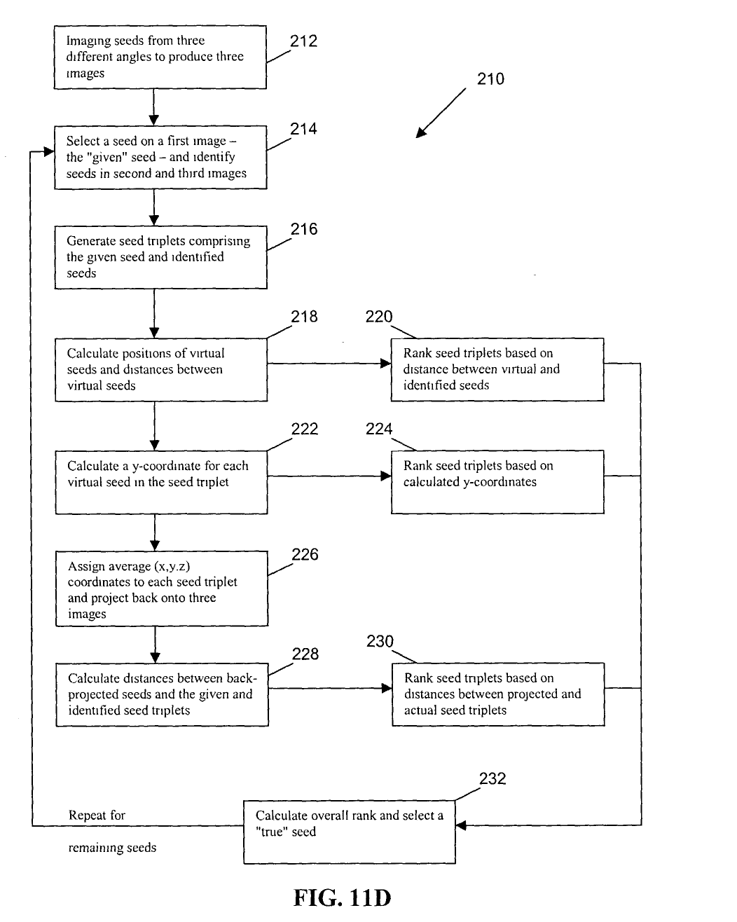

FIGS. 11A-11C show films used in the assignment of seed triplets. FIG. 11D is a flowchart 210 depicting the following method. The algorithm works in the following manner:

a) Seeds are segmented (i.e. detected) in any random order on the three films 180, 182, and 184.

b) A seed 186 is selected 214 in the first film 180; seeds in the other two views 182, 184 that are within an interval of Dy=0.75 cm (represented by dotted lines 188) from this seed 186 are chosen 214 as possible candidates.

c) From seed 186 and the possible candidate seeds, seed triplets (sets containing one seed from each view) are generated 216. For the illustrated example, say seed 186 from image 180, seed 192 from image 182 and seed 194 from image 184 form a subject seed triplet. For each such triplet the position of the virtual seeds that would have generated these 2D seed images (i.e. the images 180, 182 and 184 of the subject triplet's seeds 186, 192 and 194 respectively) is calculated 218. Generally, a triplet will result in three virtual seeds (see FIG. 10B) either because the triplet does not correspond to a real seed or because of errors in the 2D coordinates mentioned above.

d) The Euclidean distances between the three virtual seeds of a triplet, projected in the plane x-z plane or plane Z,O,X (where y=0) (See FIG. 10A) are calculated 218 and triplets sorted 220 accordingly—from the smallest distance (the best) to the largest. This is the first sorting criterion; the first 20 triplets are retained and ranked (1st ranking).

e) For the second sorting criterion, step 222 of FIG. 11D calculates 222 the y-coordinate of each of the 20 selected triplets. For each triplet there are three different y coordinates corresponding to each of the three virtual seeds of the triplet. A new ranking is produced 224 (2nd ranking) based on the standard deviation of the three values. Under the assumption that a “true” triplet describing a real seed would ideally have three identical y values computed as described above, the triplets will be placed higher in the ranking as their standard deviation of y coordinates becomes smaller.

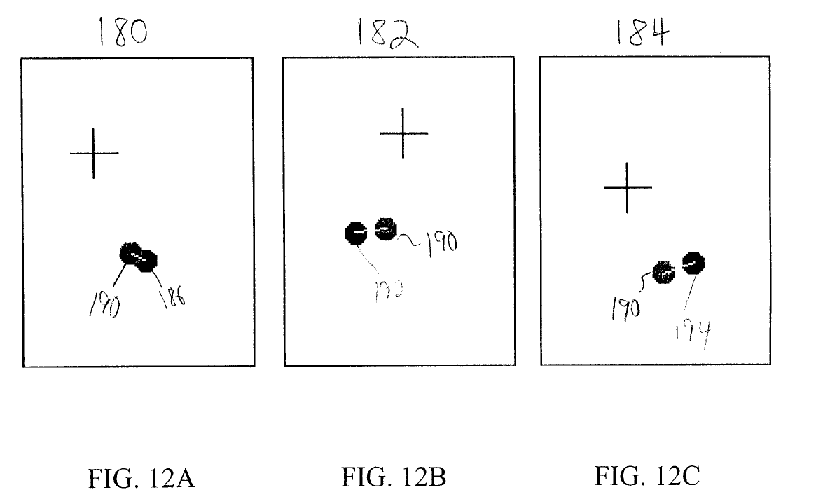

f) Each triplet is assigned 226 the average of the (x,y,z) coordinates of the three virtual seeds corresponding to the triplet. That is, the average x coordinate among the three virtual seeds is calculated, the average of the y coordinates of the three virtual seeds and the average of the z coordinates of the three virtual seeds are calculated. The resulting average x, y, z coordinates define a corresponding average virtual seed which is back-projected 228 on the three films 180, 182, 184. The Euclidean distance between reconstructed (average virtual seeds 190 back-projected to films 180, 182 and 184) and digitized seeds 186, 192, 194 is used for the third ranking 230 of the triplets; the smaller the sum of the three distances, the higher the triplet will be ranked. FIGS. 12A-12C are illustrative.

g) The triplet with the best overall rank 232 (i.e. near highest in each of the first, second and third ranks) is selected to represent a “true” seed.

This process is repeated sequentially for all seeds in the first film 180 of FIG. 11A. The triplets thus obtained may (and often do) contain seeds from images 182 and 184 that are used more than once; similarly there may be imaged seeds that are not used at all. This group of seeds must be re-examined and redistributed in triplets. Steps b through g are repeated for these seeds, with the difference that, at step b, the initial seed is taken from the second image 182.

Transformation of Implanted Seed Coordinates from the Patient Coordinate System to the Planning Coordinate System:

Contours of the prostate and other structures are outlined by the physician in the ultrasound coordinate system (UCS), a coordinate system referenced with respect to the ultrasound probe. To obtain real-time dosimetric information during the implantation procedure and—if needed—to modify or re-optimize the treatment plan, it is necessary to transfer information about seed locations from the patient coordinate system, for example the fluoroscopy image coordinate system (FCS), back to the planning coordinate system, for example, UCS. To fuse the two spaces (coordinate systems), a modified ultrasound probe unit that has a set of non-coplanar radio-opaque markers (reference points) imbedded in it, and which can be identified both in the FCS and in the UCS is used. This uniquely determines the translation-rotation transformation between the two coordinate systems.

Starting with the markers' coordinates in the two spaces (FCS and UCS), a general transformation (translation-rotation) that optimally matches the two sets of coordinates must be found. This is not a simple Euclidean transformation. Firstly, the magnification of the fluoroscopic images is uncertain. Secondly, because the markers' coordinates are unavoidably subject to experimental errors, the transformation must be optimal in the least-squares sense. Thus, the algorithm described below represents an affine transformation.

Translation and scaling are, respectively, additive and multiplicative processes. To find a representation that allows one to combine any arbitrary sequence of such transformations it is appropriate to represent points, (x, y, z), as four-dimensional vectors, (wx, wy, wz, w), where w≠0. This is called the homogeneous form. (The original representation is obtained if one divides by w and the value w=1. Such makes the correspondence between the 3D and 4D representations transparent).

The translation elements are [tₓ,t_{y},t_{z},0] and thus the translation matrix T can be arranged to be:

$$T = [ \begin{matrix}1 & 0 & 0 & 0 \\ 0 & 1 & 0 & 0 \\ 0 & 0 & 1 & 0 \\ {t}_{x} & {t}_{y} & {t}_{z} & 1\end{matrix} ] ;$$

Rotations can be either described with respect to the Cartesian axes, for instance:

$$Rz = [ \begin{matrix}cos α & sin α & 0 & 0 \\ - sin α & cos α & 0 & 0 \\ 0 & 0 & 1 & 0 \\ 0 & 0 & 0 & 1\end{matrix} ] ;$$

in the case of a rotation about the z axis, or with respect to a general axis

$$R = [ \begin{matrix}t {x}^{2} + c & t x y + s z & t x z - s y & 0 \\ t x y - s z & t {y}^{2} + c & t yz + s x & 0 \\ t xz + sy & t yz - sx & t {z}^{2} + c & 0 \\ 0 & 0 & 0 & 1\end{matrix} ] ;$$

where c=cos θ; s=sin θ; t=1−cos θ=1−c; and [x,y,z] is a unit vector on the axis of rotation, and θ the angle of rotation.

The transformation P_{B}→R (p_{A}) where p=[x y z 1]; pεA and P=[X Y Z 1]; PεB, can be represented as:

$$[ XYZ1 ] = [ xyz1 ] [ \begin{matrix}xx & yx & zx & 0 \\ xy & yy & zy & 0 \\ xz & yz & zz & 0 \\ {t}_{x} & {t}_{y} & {t}_{z} & 1\end{matrix} ] = [ xyz1 ] \cdot R ;$$

and thus the problem at hand is to find the transformation R. The minimum number of markers thus needed is four. In the preferred embodiment, five non-coplanar markers are used to minimize errors.

Using the known marker coordinates in the two coordinate spaces at hand, the following system of equations are obtained:

$$[ \begin{matrix}{X}_{1} & {Y}_{1} & {Z}_{1} & 1 \\ {X}_{2} & {Y}_{2} & {Z}_{2} & 1 \\ \cdot & \cdot & \cdot & \cdot \\ {X}_{7} & {Y}_{7} & {Z}_{7} & 1\end{matrix} ] = [ \begin{matrix}{x}_{1} & {y}_{1} & {z}_{1} & 1 \\ {x}_{2} & {y}_{2} & {z}_{2} & 1 \\ \cdot & \cdot & \cdot & \cdot \\ {x}_{7} & {y}_{7} & {z}_{7} & 1\end{matrix} ] \cdot R$$

This is equivalent to the general form b=Ax. The corresponding least square problem is to find a vector x such that ∥Ax−b∥² is minimized. The singular value decomposition approach is employed here to solve this problem, as the method is computationally stable and does not require a special assumption on matrix A. Mathematically,

A=UΣV^{T}

where U(m,m) and V(n,n) are orthogonal matrices, and Σ is a diagonal matrix with elements σᵢ,[i=1 . . . min(m, n)],σ₁>σ₂> . . . σ_{min(m,n)}≧0. The optimal solution for the least square problem is given as:

x=VΣ⁻¹U^{T}b

In order to perform this transformation quickly—and with minimal interference with the patient—use is made of the fixed geometry of the ultrasound probe and the reproducibility of its position. Specifically, the needle template (an accessory attached to the ultrasound probe during the implantation procedure) and implicitly the ultrasound probe define the ultrasound space (Ultrasound Coordinate System). Radio-opaque markers are attached to the ultrasound probe at known positions with respect to the ultrasound guiding needle template. Therefore, by identifying the points in the fluoroscopy images, one can reconstruct their positions in the FCS and uniquely define the transformation to the UCS, as discussed here.

Validation of Seed Coordinate Reconstruction and Coordinate Transformation Tools:

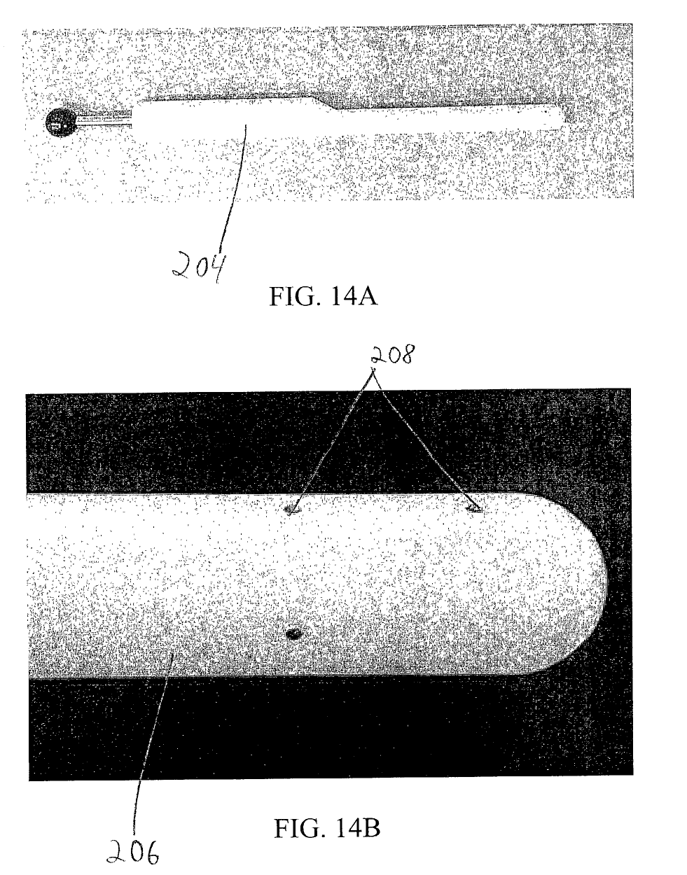

To validate the seed coordinate reconstruction algorithm and the coordinate transformation tools described above, a custom pelvic phantom 200 (CIRS Inc, Norfolk Va.) that contains a collection of dummy seeds 130 of FIG. 5 (model 6711—Amersham Corp. Arlington Heights Ill.) within a simulated prostate gland is used. FIG. 13 is a perspective view of the pelvic phantom 200. Pelvic bone, bladder, urethra and rectum are also built in the phantom 200. The ultrasound probe 204 (FIG. 14A) is placed in the ‘rectal space’ 202 of the phantom 200. A CT scan of the phantom/probe 200, 204 is performed and the positions of the dummy seeds 130 and the x-ray markers 208 on the probe 204 are determined using a commercial post implant analysis software package (INTERPLANT V.1.0, Burdette Medical Systems, Champaign Ill.).

FIGS. 14A and 14B are schematic views of a prototype ultrasound probe 204 and an enlargement of the tip 206 of the probe, respectively. A set of three fluoroscopic images of the same system are also taken with the ultrasound probe 204 remaining in the same position in the phantom 200. The X-ray markers 208 and dummy seeds 130 are identified and their positions reconstructed. Using the X-ray markers 208 set of coordinates as a guide, the seeds coordinates are then transformed into CT space that is equivalent to the UCS. The match between the transformed seeds coordinates will be checked against the coordinates detected on the CT. Since seeds are not clearly visible in ultrasound images, the validation of the system is done with CT instead.

Real-Time Re-Optimization Algorithm in IDDO Planning:

The optimization treatment-planning module used for IDDO employs the integer programming (IP) technique. Data for a patient include discretized representations of slices of the tumor site (the diseased prostate) and neighboring healthy organs; and dose-volume restrictions for various anatomical structures. A grid of potential seed positions and exposure rate constants for the radioactive sources are also recorded during the actual implant procedure. Let qⱼ be a binary value indicator for recording occupancy of grid position j with a seed (qⱼ=0 or 1). Then the total dose at point P is

$${\sum }_{j = 1}^{n} D ( ‖ P - {Q}_{j} ‖ ) {q}_{j} \qquad ( 1 )$$

where Qⱼ is a vector corresponding to the coordinates of grid point j, ∥•∥ denotes the Euclidean norm, D(r) denotes the dose contribution of a seed to a point r units away, and n denotes the total number of possible seed positions. The target dose levels, say L_{P} and U_{P}, for the dose at point P can be incorporated within Eq(1) to form the dosimetric constraints for the model

$${U}_{P} \geq {\sum }_{j = 1}^{n} D ( ‖ P - {X}_{j} ‖ ) {x}_{j} \geq {L}_{P} . \qquad ( 2 )$$

Values of L_{P} and U_{P} within the target can vary according to the desired dose-volume distribution input by physician. In particular, dose received by urethra and rectum will be strictly controlled. The IP-based treatment-planning module provides a very flexible planning environment for simultaneously incorporating multiple physical constraints within the planning process. A preliminary implementation of this treatment planning system has been used successfully in a computerized treatment planning study (Lee et al, 1999), a recent MRS-guided dose escalation study (Zaider et al, 2000), and a continuous dose control investigation for multi-period treatment planning (Lee and Zaider, 2001).

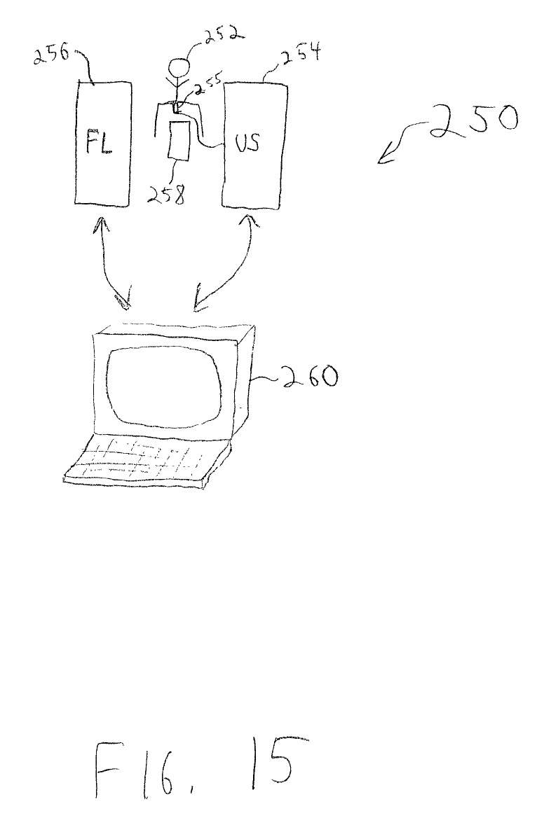

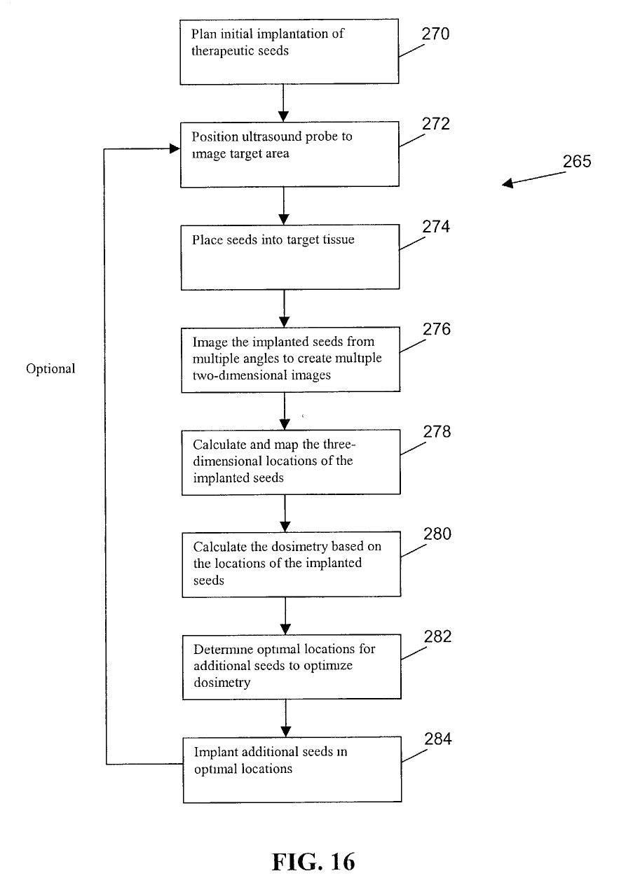

According to the foregoing techniques and algorithms (mathematical equations) for seed reconstruction, transformation from seed reconstruction space (FCS) to seed implanting/medical procedure guiding space (UCS), and dose optimization-reoptimization, a computerized system embodying the present invention is presented in FIGS. 15 and 16. FIG. 15 is a schematic view 250 of an operating room in which the methods of the preferred embodiment are implemented and FIG. 16 is a flowchart 265 describing the overall operative procedure of the preferred embodiment.

A computer system 260 is used initially to plan 270 the implantation of therapeutic seeds into a patient 252. An ultrasound imaging system 254 is used to image 272 the target area in a planning coordinate system. This may be done with an ultrasound probe 255 in the patient's 252 rectum A fluoroscopic imaging system 256 is also present for producing images in a patient coordinate system. Seeds are placed 274 in the target tissue using known methods and instruments 258 for brachytherapy seed insertion. An image is then taken 276 of the implanted seeds from multiple angles (at least three in the preferred embodiment) to produce multiple two-dimensional images. The three-dimensional locations of the implanted seeds is then calculated and mapped 278. From the locations of the implanted seeds, the resulting dosimetry is then calculated 280. From the resulting dosimetry, optimal locations for the implantation of additional seeds is then determined 282 for producing an optimal dosimetry. Finally, the additional seeds are then implanted 284 in the optimal locations. This process may be repeated to continually assess and optimize dosimetry of the implanted therapeutic seeds.

In the preferred embodiment, extra modules interact with the seed reconstruction and coordinate transformation engines to facilitate the calculation of radiation dose received intermittently during the implantation process for the purpose of re-optimization.

During implantation, an optimal treatment plan is first obtained based on initial operation-acquired ultrasound images using the real-time treatment planning system (Lee, 1999). For implementation of re-optimization in IDDO, three re-optimization steps will be performed. The input for re-optimization consists of: 1) the position of seeds already deposited in the prostate, which will provide the current dose distribution received by the gland thus far; 2) possible remaining positions for further implants; 3) dose bounds for various anatomical structures; 4) coverage and conformity requirement; and 5) strict dose limits for urethra and rectum. The first re-optimization will be performed when the number of seeds implanted is ½ of the total number of seeds in the original plan. When all the seeds determined by the first re-optimization are implanted, a second re-optimization will be performed to assist in eliminating possible cold spots. For each re-optimization, strict dose bounds of 100% and 120% of the prescription dose are imposed on the urethra. Also, 100% coverage to the prostate volume is desired.

The sensitivity and effect of the procedure with respect to the total number of re-optimization iterations required will be analyzed. A simulation procedure can be employed to consider various scenarios of seed distribution as a result of the order in which seeds are implanted. For each scenario, a three-way comparison will be performed: (a) initial optimized plan (obtained during real-time planning in the operating room), (b) implemented plan, and (c) IDDO plan. Post-implant dose analysis, coverage and conformity measures, as well as dose received by urethra and rectum will be used to gauge the results.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for implanting therapeutic seeds in target tissue, comprising the steps of:
(a) placing a first set of therapeutic seeds in the target tissue such that the therapeutic seeds are implanted seeds;
(b) imaging the implanted seeds in the target tissue in a manner that produces two-dimensional images of the implanted seeds;
(c) using computer processing: determining the three-dimensional locations of the implanted seeds based on the two-dimensional images;
calculating dosimetry of the implanted seeds based on the determined three-dimensional locations of the seeds in the target tissue; and
determining optimal locations in the target tissue for additional seeds based on the calculated dosimetry of the implanted seeds, such optimal locations optimizing total dosimetry of additional seeds and the implanted seeds combined in the target tissue; and
(d) implanting additional seeds in the optimal locations.

2. The method of claim 1 wherein the step of imaging includes employing ultrasound imaging.

3. The method of claim 1 wherein the step of imaging includes employing fluoroscopic imaging.

4. The method of claim 1 wherein the step of imaging includes employing magnetic resonance imaging.

5. The method of claim 1 wherein the step of imaging the implanted seeds in the target tissue further comprises the step of obtaining at least three two-dimensional images of the implanted seeds, each two-dimensional image being from a different angle.

6. The method of claim 5 wherein the step of determining three-dimensional locations of the implanted seeds further comprises evaluating projections of the implanted seeds on at least three two-dimensional images of the implanted seeds.

7. The method of claim 1 further comprising the step of translating seed locations from a patient coordinate system to a planning coordinate system.

8. The method of claim 7 wherein the patient coordinate system is a fluoroscopy imaging coordinate system and the planning coordinate system is an ultrasound imaging coordinate system used in ultrasound imaging of the target tissue; and
the step of translating includes locating lead markers on an ultrasound probe used in the ultrasound imaging of the target tissue.

9. The method of claim 8 wherein the step of translating further comprises the steps of:
(i) identifying at least two common markers in each the patient coordinate system and the planning coordinate system;
(ii) in the planning coordinate system, mechanically measuring the markers' coordinates with respect to a template;
(iii) in the patient coordinate system, reconstructing the markers' measurements from their two-dimensional projections; and
(iv) calculating a general transformation such that, when applied to the markers, will result in the superposition of the two sets of markers.

10. The method of claim 1 wherein steps (a) through (d) occur within a single operative session.

11. The method of claim 1 wherein:
the step of imaging includes imaging the implanted seeds in the target tissue from at least three different angles to produce first, second and third two-dimensional images of the seeds; and
the step of determining three-dimensional locations of implanted seeds includes
(i) for a given seed in the first image, identify seeds in the second and third images that are within an interval Dy from the given seed;
(ii) generating seed triplets comprised of the given seed in the first image, an identified seed in the second image and an identified seed in the third image;
(iii) for each seed triplet, calculating positions of virtual seeds for generating the first, second and third images of the seeds in the triplet;
(iv) determining distances between the calculated positions of the virtual seeds, said determining including projecting calculated positions of virtual seeds into an x-z plane where y=0;
(v) using the calculated positions of the virtual seeds and x, y, z coordinates of the virtual seeds, ranking the seed triplets relative to each other;
(vi) based on ranking, selecting a seed triplet to represent the given seed; and
(vii) repeating steps (i) through (vi) at least for each seed in the first image.

12. The method of claim 11 wherein the step of repeating includes using a seed in the second image as the given seed and identifying seeds in the first and third images to generate seed triplets.

13. A method for detecting the location of therapeutic seeds in subject tissue comprising the computer implemented steps of:
(a) imaging therapeutic seeds in subject tissue from at least three different angles to produce first, second and third images of the seeds;
(b) for a given seed in the first image, generating seed triplets comprised of the given seed, an identified seed in the second image and an identified seed in the third image;
(c) for each seed triplet, calculating positions of virtual seeds for generating the first, second and third images of the seeds in the triplet;
(d) determining distances between the calculated positions of the virtual seeds, said determining including projecting calculated positions of virtual seeds into an x-z plane where y=0;
(e) using the calculated positions of the virtual seeds and x, y, z coordinates of the virtual seeds, ranking the seed triplets relative to each other;
(f) based on ranking, selecting a seed triplet to represent the given seed; and
(g) repeating steps (b) through (f) at least for each seed in the first image.

14. The method of claim 13 wherein step (b) includes identifying seeds in the second and third images that are within an interval Dy from the given seed.

15. The method of claim 13 wherein step (d) includes:
determining distances between the calculated positions of virtual seeds, said determining including projecting calculated positions of virtual seeds into an x-z plane where y=0; and
sorting and ranking the seed triplets based on the determined distances, the smallest distances ranking highest.

16. The method of claim 15 wherein step (d) further includes:
for each of certain seed triplets, calculating a y-coordinate for each virtual seed in the triplet and ranking the seed triplets based on the calculated y-coordinates.

17. The method of claim 16 wherein step (d) further includes:
(i) assigning to each of certain seed triplets a respective average virtual seed defined by an average of x, y, z coordinates of the virtual seeds of the triplet;
(ii) projecting the respective average virtual seeds back on to the first, second and third images;
(iii) measuring the distances between the respective average virtual seeds of seed triplets and the respective given and identified seeds of the seed triplets; and
(iv) ranking the seed triplets based on smallest measured distance.

18. The method of claim 13 wherein step (d) includes providing multiple rankings of the seed triplets, and step (e) includes selecting a seed triplet based on overall ranking of the seed triplet.

19. The method of claim 13 further comprising the step of translating seed locations from a patient coordinate system to a planning coordinate system.

20. The method of claim 19 wherein the step of translating includes:
(i) identifying at least two common markers in each the patient coordinate system and the planning coordinate system;
(ii) in the planning coordinate system, mechanically measuring the markers' coordinates with respect to a template;
(iii) in the patient coordinate system, reconstructing the markers' measurements from their two-dimensional projections;
(iv) calculating a general transformation such that, when applied to the markers, will result in the superposition of the two sets of markers.
